(19) United States
(12) Patent Application Publication
(10) Pub. No.: US 2025/0186643 A1
(43) Pub. Date: Jun. 12, 2025

(54) NEGATIVE PRESSURE CONTAINMENT SYSTEM AND METHOD

(71) Applicant: Cory William Chittenden, Montmartre (CA)

(72) Inventor: Cory William Chittenden, Montmartre (CA)

(21) Appl. No.: 18/530,366

(22) Filed: Dec. 6, 2023

(51) Int. Cl. A61L 2/24; A47L 9/20

(52) U.S. Cl. CPC A61L 2/24; A47L 9/20; A61L 2202/25; A61L 2209/14; A61L 2209/20

(57) ABSTRACT

A negative pressure enclosure includes a containment cavity that can be suctioned onto a surface to prevent particles, such as dust particles, from infecting patients within a health care environment during work, such as repair or maintenance work. Particularly, the negative pressure enclosure couples with a vacuum pump to create the negative pressure within the containment cavity; and with a HEPA vacuum to suction particles directly from the containment cavity during the work, preventing the particles from entering the environment external to the negative pressure enclosure.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of debris collecting systems of existing art and more specifically relates to an enclosure that contains and collects particles, such as dust particles, thereby maintaining an uncontaminated environment external to the enclosure.

### RELATED ART

In a healthcare or other sterilized environment, various maintenance tasks may need to be performed to ensure the overall functionality and appearance of the facility. These tasks can include drywall repair, painting, outlet maintenance, maintenance of walls and flooring, etc. However, maintaining a sterile environment in healthcare facilities is of paramount importance to ensure the safety and well-being of patients. This includes not only the patient care areas but also any spaces where maintenance work is conducted. Contractors and maintenance staff must follow strict protocols to prevent contamination and uphold the highest standards of cleanliness.

Ensuring the sterility of the area can be a significant challenge. Currently, the affected area is typically evacuated, and it must undergo thorough cleaning and sterilization before it can be used again. While this protocol is essential for patient safety, it can be a nuisance and a significant disruption for both the medical staff and patients. Evacuating a workspace may lead to rescheduling appointments, shifting healthcare operations, and causing inconveniences. Accordingly, a suitable solution is desired.

### SUMMARY OF THE INVENTION

In view of the foregoing disadvantages inherent in the known debris collecting enclosure art, the present disclosure provides a novel negative pressure enclosure for maintaining an uncontaminated environment. The general purpose of the present disclosure, which will be described subsequently in greater detail, is to provide an enclosure that maintains an uncontaminated environment in a sterile area, such as a healthcare facility. The enclosure suctions to a surface (such as a wall), surrounding a work area in the sterile area, and simultaneously prevents escape of particles produced during the work whilst suctioning the particles from the enclosure via a HEPA (High-Efficiency Particulate Air) vacuum.

A negative pressure enclosure is disclosed herein. As above, in some embodiments, the negative pressure enclosure may be particularly used in a healthcare environment. The negative pressure enclosure may include an enclosure, a rear panel, a sealing portion, a vacuum pump port, and a vacuum hose coupler. The enclosure may include an outer shell defining an interior containment cavity. For example, the outer shell may include a front side opposite a rear side including an outwardly extending edge, a top side opposite a bottom side and at least two side walls. Further, the enclosure may include a first opening at a rear side of the enclosure and at least one arm opening disposed within a front side of the enclosure providing access to the interior containment cavity, thereby enabling a user to perform work within the interior containment cavity of the enclosure by inserting at least one arm into the at least one arm opening.

The rear panel may be attached to the rear side of the enclosure and may include a second opening aligned with the first opening. The sealing portion may include a first seal bordering the second opening and a second seal bordering the rear panel.

The vacuum pump port may be disposed through at least the rear panel and configured to receive a connector end of an air-line. The air-line is configured to connect at an opposite end to a vacuum pump and, upon actuation of the vacuum pump, a negative pressure is created in the interior containment cavity, thereby pushing the sealing portion against a surface to seal the enclosure thereto. In some embodiments, the vacuum pump port may be a quick-connect.

The hose coupler may be disposed in a side of the enclosure and spanning through to the interior containment cavity. The hose coupler may be configured to receive an end of a HEPA vacuum hose therein and the HEPA vacuum hose connects to a HEPA vacuum at another opposite end thereof. Upon actuation of the HEPA vacuum, particles (such as dust and other debris) produced during the work is suctioned from the interior containment cavity via the HEPA vacuum hose, thereby maintaining an uncontaminated environment external to the enclosure.

According to another embodiment, a method of containing and collecting particles produced during work is also disclosed herein. The method may include the steps of: providing the negative pressure enclosure as above; connecting a connector end of an air-line to the vacuum pump port, an opposite end of the air-line being connected to a vacuum pump; turning the vacuum pump on; placing the enclosure against a surface where work is to be performed; pushing the enclosure against the surface until the enclosure is held against the surface via a negative pressure created in the interior containment cavity by the vacuum pump; inserting an end of a HEPA vacuum hose into the vacuum hose coupler, an opposite end of the HEPA vacuum hose being connected to a HEPA vacuum; turning the HEPA vacuum on; inserting at least one arm through the at least one arm opening; and performing said work within the interior containment cavity, particles produced during said work being suctioned from the interior containment cavity via the HEPA vacuum hose, thereby maintaining an uncontaminated environment external to the enclosure.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a negative pressure enclosure for maintaining an uncontaminated environment, constructed and operative according to the teachings of the present disclosure.

FIG. 1 is a side perspective view of a negative pressure enclosure in use, according to an embodiment of the disclosure.

FIG. 2 is a front perspective view of the negative pressure enclosure held against a surface and attached to a HEPA vacuum hose and a vacuum pump air-line, according to an embodiment of the disclosure.

FIG. 3 is a perspective view of a bottom side of the negative pressure enclosure including a hose coupler, according to an embodiment of the present disclosure.

FIG. 4 is a perspective view of a front side of the negative pressure enclosure including a pair of arm openings, according to an embodiment of the present disclosure.

FIG. 5 is a perspective view of a rear side of the negative pressure enclosure including a sealing portion, according to an embodiment of the present disclosure.

FIG. 6 is a close-up view of the rear side of the negative pressure enclosure including the sealing portion and an aperture for a vacuum pump port, according to an embodiment of the present disclosure.

FIG. 7 is a close-up view of the front side of the negative pressure enclosure including the vacuum pump port, according to an embodiment of the present disclosure.

FIG. 8 is a perspective view of a bottom side of a negative pressure enclosure, according to another embodiment of the present disclosure.

FIG. 9 is a perspective view of a front side of the negative pressure enclosure, according to another embodiment of the present disclosure.

FIG. 10 is a perspective view of a rear side of the negative pressure enclosure, according to another embodiment of the present disclosure.

FIG. 11 is a flow diagram illustrating a method of containing and collecting particles produced during work, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a debris collecting enclosure. Generally, the debris collecting enclosure may include a containment cavity that can be ‘suctioned’ onto a surface to prevent particles, such as dust, from infecting patients within a hospital, doctor's office, and the like; and uses a HEPA (High-Efficiency Particulate Air) filtered vacuum to suction the particles from the containment cavity. As such, the enclosure is designed to assist with maintenance and renovations in a highly acute health care environment and enables hands free construction in small areas without taking the acute care room out of commission for a prolonged period of time.

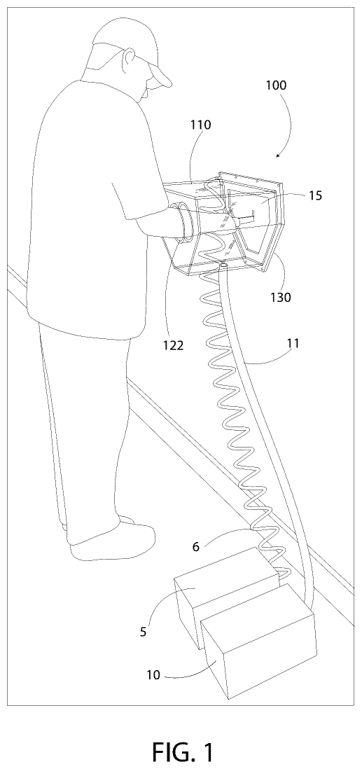

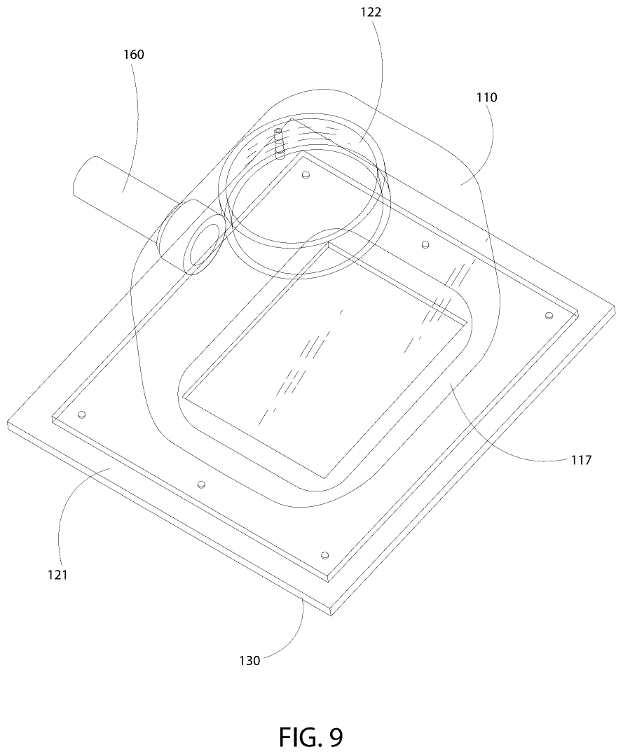

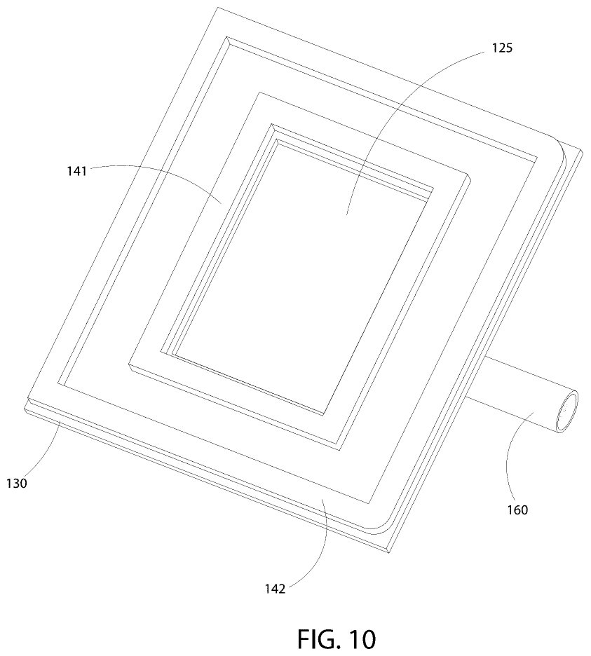

Referring now more specifically to the drawings by numerals of reference, there is shown in FIGS. 1-10, various views of a negative pressure enclosure 100.

Referring first to FIG. 1, there is shown a side perspective view of the negative pressure enclosure 100 in use. As shown here, the negative pressure enclosure 100 is particularly used for containing and collecting particles produced during work, such as repair or maintenance work, that can be conducted within the negative pressure enclosure 100. As above, the negative pressure enclosure 100 is preferably and most suitably adapted for use in sterile environments, such as hospitals, doctor's offices, and the like.

As shown in FIG. 1 in particular, the negative pressure enclosure 100 may include an enclosure 110 defining an interior containment cavity 111 (i.e., the area in which the user performs their work, such as [but not limited to] dry wall repair, as shown here). As such, the enclosure 110 is preferably entirely, or at least substantially, transparent so as to enable the user to see through the enclosure 110. For example, in some embodiments, the enclosure 110 may be constructed from (but is not limited to) glass, acrylic, plastic, or the like.

As shown here, the negative pressure enclosure 100 is configured for use with a vacuum pump 5 to create a pressure differential (negative pressure within the enclosure 110) and ‘suction’ the negative pressure enclosure 100 to a surface 15 (i.e., the negative pressure causing the higher atmospheric pressure to push the negative pressure enclosure 100 against the surface 15, holding it thereto); and a HEPA (High-Efficiency Particulate Air) vacuum 10 to suction the particles (such as dust and other debris) from the negative pressure enclosure 100.

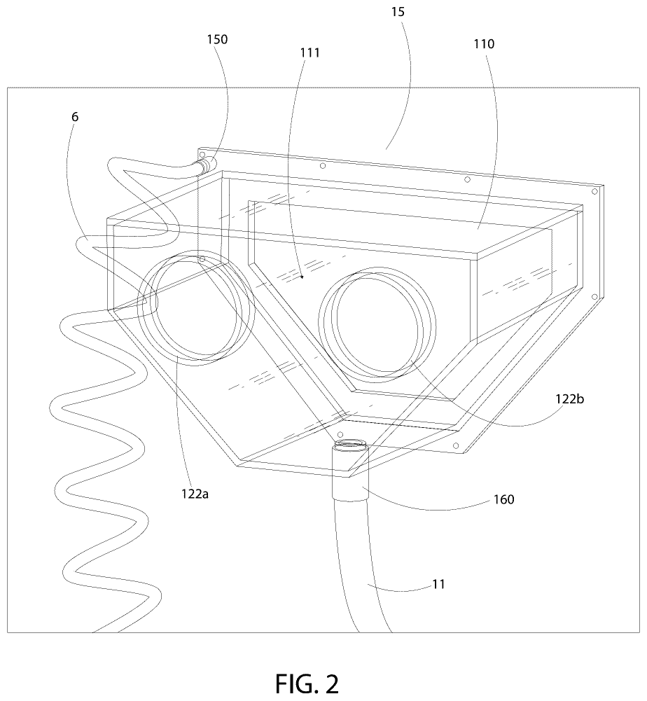

Referring now also to FIG. 2 with reference still to FIG. 1, there is shown a front perspective view of the negative pressure enclosure 100. As shown here, the enclosure 110 may include (but is not limited to) two arm openings 122 a, 122 b. Particularly, the arm openings 122 a, 122 b may be disposed within a front side 115 of the enclosure 110. As shown, the arm openings 122 a, 122 b provide access to the interior containment cavity 111, thereby enabling the user to perform the work within the interior containment cavity 111 of the enclosure 110 by placing their arms through the arm openings 122 a, 122 b and into the enclosure 110 (one arm per arm opening 122 a, 122 b). As shown here, the arm openings 122 a, 122 b are preferably circular in configuration, so as to comfortably accommodate the arms of the user.

As above, the negative pressure enclosure 100 is particularly configured to suction to a surface 15 (again, by creating negative pressure within the enclosure 110). For example, as demonstrated in FIG. 1, the negative pressure enclosure 100 may be configured to suction to a wall.

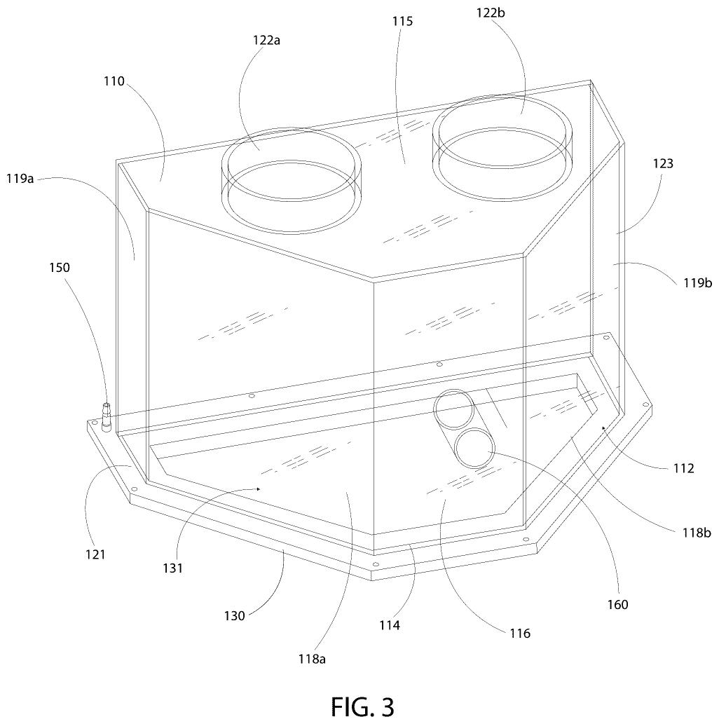

Referring now also to FIGS. 3-7, with reference still to FIGS. 1-2, there are shown various views of the negative pressure enclosure 100 according to one or more embodiments of the present disclosure. Referring first to FIGS. 3-5, illustrating perspective views of the negative pressure enclosure 100, there is shown a first opening 112 disposed within a rear side 114 of the enclosure 110 (the rear side 114 being opposite the front side 115 of the enclosure 110).

Further, a rear panel 130 may be attached to the rear side 114 and may include a second opening 131 aligned with the first opening 112 (i.e., sharing a central axis) and together forming a rear opening 125 of the negative pressure enclosure 100. As can be seen particularly in FIGS. 1-7, in some embodiments, the enclosure 110 may include an irregular hexagonal prism shape. For example, as shown, the enclosure 110 may include an outer shell 123 comprising the front side 115, or front wall, including an irregular hexagonal shape face, a bottom side 116, or bottom wall, including a rectangular face, two opposing lower side walls 118 a, 118 b including rectangular faces, two opposing upper side walls 119 a, 119 b also including rectangular faces and a top side 117, or top wall, including a rectangular face having a length generally perpendicular to a length of the bottom side 116.

It is contemplated that each wall of the enclosure 110 is constructed to provide maximum workspace for the user, whilst maintaining durability and sturdiness needed to withstand the negative pressure within the interior containment cavity 111, work performed inside, pressure exerted by the user's arms on the enclosure 110, etc.

The rear side 114 of the enclosure 110 may be defined entirely by the first opening 112. Accordingly, the first opening 112 may include an irregular hexagonal shape, equal to the front side 115 of the enclosure 110. In some embodiments, the rear panel 130 may also include an irregular hexagonal shape, with the second opening 131 thereof also including the irregular hexagonal shape. The second opening 131 of the rear panel 130 may be smaller in size than the first opening 112 of the enclosure 110.

The rear panel 130, as the element that contacts the surface 15 to which the negative pressure enclosure 100 is being attached, may be flat in profile, so as to aid in suctioning of the negative pressure enclosure 100 to the surface 15.

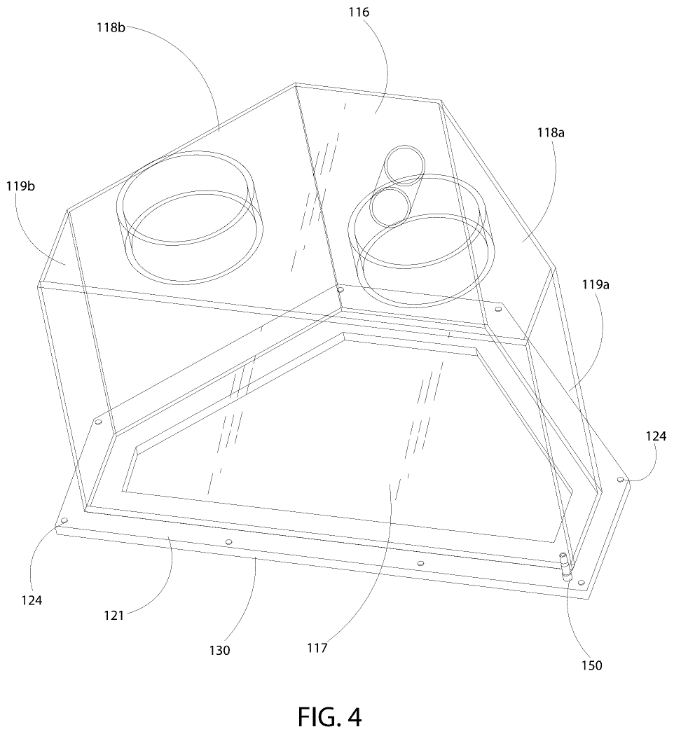

Further, in some embodiments, the rear side 114 of the enclosure 110 may include an outwardly extending edge 121. This outwardly extending edge 121 attach directly to the rear panel 130. Particularly, as shown in FIG. 4, fasteners 124 such as (but not limited to) rivets may be used to attach the outwardly extending edge 121 to the rear panel 130 at each corner of the rear panel 130 and at equally spaced locations along a top edge of the rear panel 130.

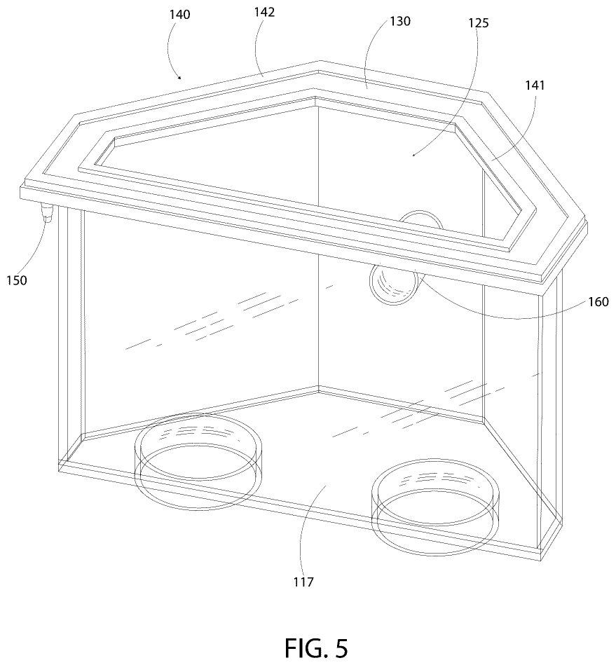

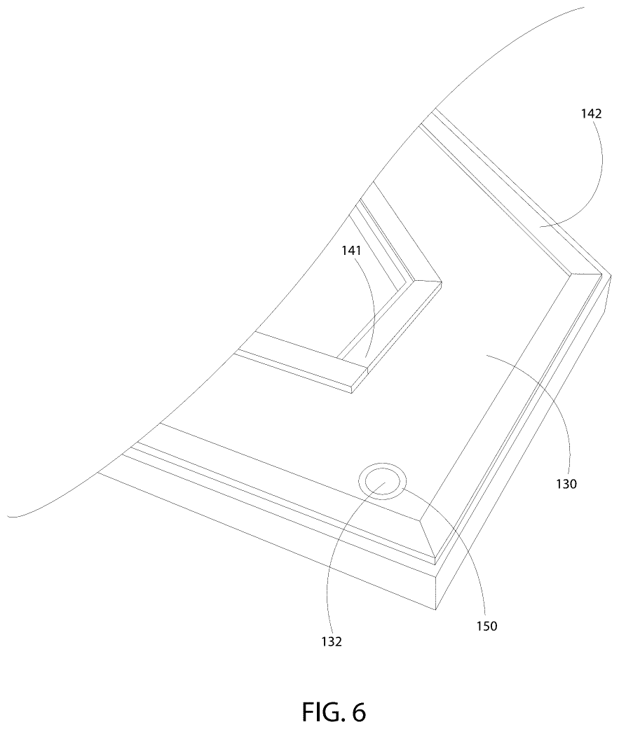

As shown more particularly in FIGS. 5-6, a sealing portion 140 may be provided at the rear side 114 of the enclosure 110. Particularly, the sealing portion 140 may include a first seal 141 bordering the second opening 131, (the rear opening 125 of the negative pressure enclosure 100) and a second seal 142 bordering the rear panel 130 (outer edge of the rear panel 130). The dual seals 141, 142 may provide an enhanced and consistent seal to the enclosure 110. In some embodiments, the first seal 141 and the second seal 142 may be (but not are not limited to) foam material.

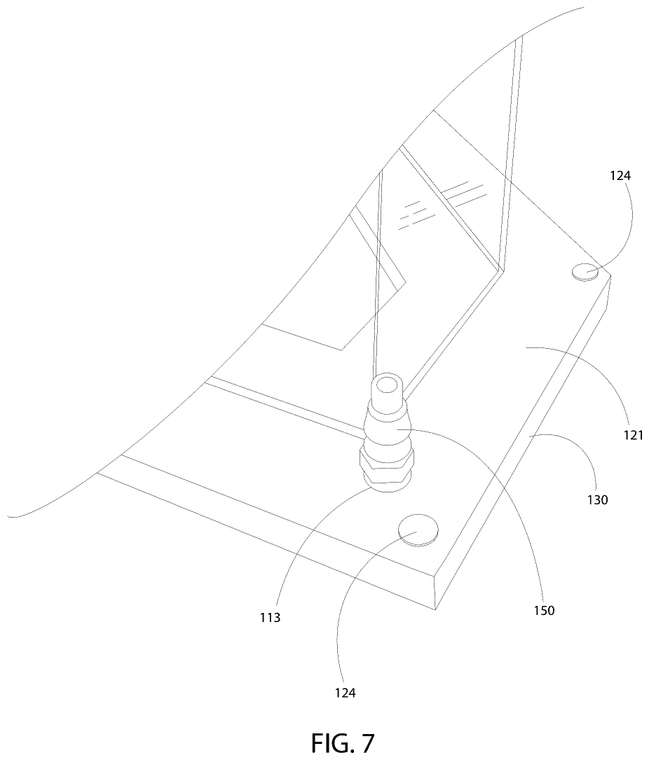

Referring now also to FIGS. 6-7, there is shown a vacuum pump port 150 disposed within the negative pressure enclosure 100, according to one or more embodiments of the present disclosure. As discussed above and shown in FIG. 1 in particular, the negative pressure enclosure 100 is configured for use with a vacuum pump 5 to create negative pressure within the enclosure 110, pushing the enclosure 110 against the surface 15 (e.g., a wall as shown in FIGS. 1-2).

As such, the vacuum pump port 150 may be configured to receive a connector end of an air-line 6. The air-line 6 may then connect at an opposite end to the vacuum pump 5 (FIG. 1) and, upon actuation of the vacuum pump 5, a negative pressure is created within the interior containment cavity 111, pushing the sealing portion 140 against the surface 15 to seal the enclosure 110 thereto. Further, the negative pressure in the enclosure 110 may prevent particles produced during the work from escaping into the environment external to the enclosure 110. In some embodiments, the vacuum pump port 150 may be a quick connect vacuum pump port 150 configured to interface with a quick connect air-line 6.

In some embodiments, the enclosure 110 may include a first aperture 113 disposed therein, and the rear panel 130 may include a second aperture 132 disposed therein and aligned with the first aperture 113. Accordingly, the vacuum pump port 150 may be disposed through first aperture 113 and the second aperture 132. For example, the vacuum pump port 150 may be disposed at a front side of the outwardly extending edge 121 at a top edge thereof (relative to the enclosure 110) and extending through to a rear side of the rear panel 130. As shown in FIG. 6 in particular, the vacuum pump port 150 may be located between the first seal 141 and the second seal 142.

A vacuum hose coupler 160 may be disposed in a side of the enclosure 110 and spanning through to the interior containment cavity 111. The vacuum hose coupler 160 may include a hollow tubular configuration configured to receive a HEPA vacuum hose 11 therein, as particularly shown in FIGS. 1-2. As discussed above and shown in FIG. 1, the HEPA vacuum hose 11 is connected to a HEPA vacuum 10 at another opposite end, and as such, particles produced during the work are suctioned through the interior containment cavity 111 via the HEPA vacuum hose 11, thereby maintaining an uncontaminated environment outside of the enclosure 110.

In some embodiments, the vacuum hose coupler 160 may be disposed in the bottom side 116 (or bottom wall) of the enclosure 110 and extending vertically and downwardly from the interior containment cavity 111, through the bottom side 116 to an exterior of the enclosure 110, enabling the HEPA vacuum hose 11 to suction the particles from below.

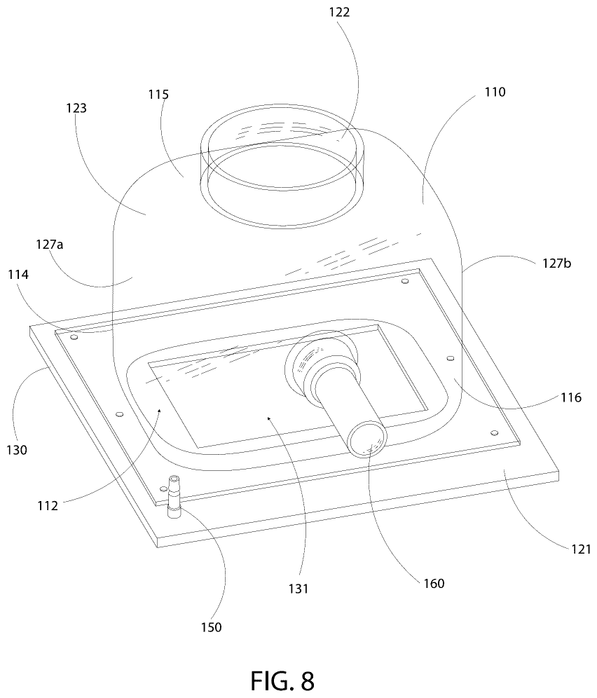

Referring now to FIGS. 8-10, there are shown various views of the negative pressure enclosure 100 according to one or more embodiments of the present disclosure. It should be appreciated that the negative pressure enclosure 100 is not limited to size, shape, configurations, etc. discussed and shown here. For example, as shown in FIGS. 8-10, instead of the hexagonal prism configuration, the enclosure 110 may include a rounded cuboid shape. In this embodiment, the rear panel 130 may include a rectangular shape. As above, the enclosure 110 may include a bottom side 116 opposite a top side 117, a rear side 114 opposite a front side 115 and two opposing side walls 127 a, 127 b.

Further, in this embodiment, one arm opening 122 may be provided at the front side 115 of the enclosure 110. This particular embodiment may be particularly useful for smaller or less complex work; whilst the embodiment shown in FIGS. 1-7 may be particularly useful for larger tasks or more detailed work requiring the use of two hands and more space.

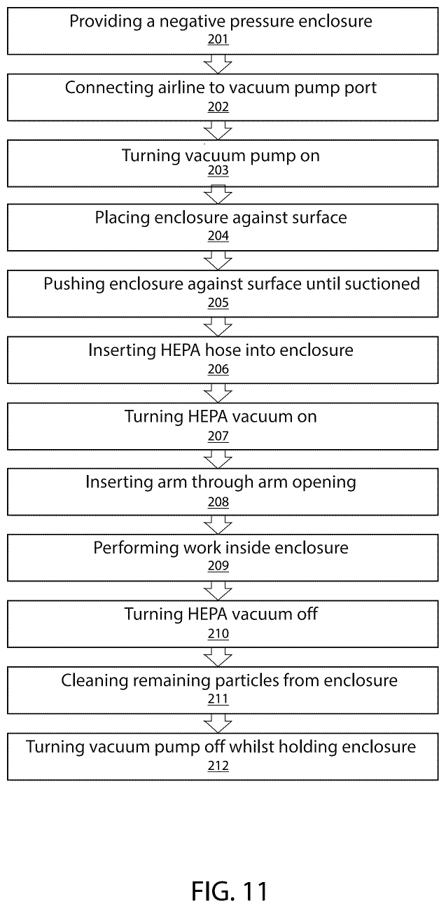

Referring now to FIG. 11 showing a flow diagram illustrating a method of containing and collecting particles produced during work (“method” 200), according to an embodiment of the present disclosure. In particular, the method 200 may include one or more components or features of the negative pressure enclosure 100 as described above.

As illustrated, the method of use 200 may include the steps of: step one 201, providing the negative pressure enclosure as above; step two 202, connecting a connector end of an air-line to the vacuum pump port, an opposite end of the air-line being connected to a vacuum pump; step three 203, turning the vacuum pump on; step four 204, placing the enclosure against a surface where work is to be performed; step five 205, pushing the enclosure against the surface until the enclosure is suctioned, or held, against the surface via a negative pressure created in the interior containment cavity by the vacuum pump; step six 206, inserting an end of a HEPA vacuum hose into the vacuum hose coupler, an opposite end of the HEPA vacuum hose being connected to a HEPA vacuum; step seven 207, turning the HEPA vacuum on, creating a negative pressure in the interior containment cavity; step eight, 208, inserting at least one arm through the at least one arm opening; and step nine 209, performing said work within the interior containment cavity, particles produced during said work being suctioned from the interior containment cavity via the HEPA vacuum hose, thereby maintaining an uncontaminated environment external to the enclosure.

Once work has been completed, further steps may be carried out in the method 200. Particularly the steps may include: step ten 210, turning the HEPA vacuum off; step eleven 211, cleaning out remaining particles left in the enclosure and on the surface; and step twelve 212, turning the vacuum pump off whilst simultaneously holding the enclosure until it comes loose from the surface (as the pressure within the enclosure 110 rises to the pressure of the environment external to the enclosure).

It should be noted that not all steps may be implemented in all cases. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for are taught herein.

It should be understood by one of skill in the art that the disclosed invention is described here in a few exemplary embodiments of many. No particular terminology or description should be considered limiting on the disclosure or the scope of any claims issuing therefrom.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the relevant patent offices and the public generally, and especially the scientist, engineers and practitioners in the art who are not familiar with patent or legal terms or phraseology, to determine quickly from a cursory inspection the nature and essence of the technical disclosure of the application.

What is claimed is:

1. A negative pressure enclosure comprising:
an enclosure including an outer shell defining an interior containment cavity and including a first opening at a rear side of the enclosure and at least one arm opening disposed within a front side of the enclosure providing access to the interior containment cavity, thereby enabling a user to perform work within the interior containment cavity of the enclosure by inserting at least one arm into the at least one arm opening;
a rear panel attached at the rear side of the enclosure and including a second opening aligned with the first opening;
a sealing portion including a first seal bordering the second opening and a second seal bordering the rear panel;
a vacuum pump port disposed through at least the rear panel, the vacuum pump port configured to receive a connector end of an air-line, the air-line configured to connect at an opposite end to a vacuum pump and wherein upon actuation of the vacuum pump, a negative pressure is created in the interior containment cavity, thereby pushing the sealing portion against a surface to seal the enclosure thereto; and
a vacuum hose coupler disposed through a side of the enclosure and into the interior containment cavity, the vacuum hose coupler configured to receive an end of a HEPA vacuum hose therein, the HEPA vacuum configured to connect to a HEPA vacuum at another opposite end thereof, and wherein upon actuation of the HEPA vacuum, particles produced during said work are suctioned from the interior containment cavity via the HEPA vacuum hose, thereby maintaining an uncontaminated environment external to the enclosure.

2. The negative pressure enclosure of claim 1, wherein the vacuum pump port is a quick connect vacuum pump port.

3. The negative pressure enclosure of claim 2, wherein the vacuum hose coupler includes a hollow tubular configuration.

4. The negative pressure enclosure of claim 3, wherein the vacuum hose coupler is disposed at a bottom side of the enclosure.

5. The negative pressure enclosure of claim 4, wherein the rear side includes an outwardly extending edge.

6. The negative pressure enclosure of claim 5, wherein the vacuum pump port is disposed through the outwardly extending edge and the rear panel.

7. The negative pressure enclosure of claim 6, wherein the vacuum pump port is located between the first seal and the second seal.

8. The negative pressure enclosure of claim 7, wherein the first seal and the second seal are constructed from a foam material.

9. The negative pressure enclosure of claim 1, wherein the enclosure includes an irregular hexagonal prism shape.

10. The negative pressure enclosure of claim 9, wherein the rear panel includes an irregular hexagon shape.

11. The negative pressure enclosure of claim 1, wherein the enclosure includes a rounded cuboid shape.

12. The negative pressure enclosure of claim 11, wherein the rear panel includes a rectangular shape.

13. A negative pressure enclosure in a healthcare environment, the negative pressure enclosure comprising:
an enclosure including a front side opposite a rear side having an outwardly extending edge, a top side opposite a bottom side and at least two side walls all defining an interior containment cavity, the rear side including a first opening at the rear side of the enclosure and at least one arm opening disposed within the front side of the enclosure providing access to the interior containment cavity, thereby enabling a user to perform work within the interior containment cavity of the enclosure by inserting at least one arm into the at least one arm opening;
a flat rear panel attached to the rear side and including a second opening aligned with the first opening;
a sealing portion including a first seal bordering the second opening and a second seal bordering the rear panel;
a quick-connect vacuum pump port disposed through the outwardly extending edge and the rear panel between the first seal and the second seal, the vacuum pump port configured to receive a quick-connect connector end of an air-line, \the air-line configured to connect at an opposite end to a vacuum pump and wherein upon actuation of the vacuum pump, a negative pressure is created in the interior containment cavity, thereby pushing the sealing portion against a surface to seal the enclosure thereto; and
a tubular vacuum hose coupler disposed in the bottom side of the enclosure and spanning through to the interior containment cavity, the vacuum hose coupler configured to receive a HEPA vacuum hose therein, the HEPA vacuum configured to connect to a HEPA vacuum at another opposite end thereof, and wherein upon actuation of the HEPA vacuum, particles produced during said work are suctioned from the interior containment cavity via the HEPA vacuum hose, thereby maintaining an uncontaminated environment external to the enclosure.

14. The negative pressure enclosure of claim 13, wherein the first seal and the second seal are constructed from a foam material.

15. The negative pressure enclosure of claim 13, wherein the enclosure includes an irregular hexagonal prism shape.

16. The negative pressure enclosure of claim 15, wherein the rear panel includes an irregular hexagon shape.

17. The negative pressure enclosure of claim 13, wherein the enclosure includes a rounded cuboid shape.

18. The negative pressure enclosure of claim 17, wherein the rear panel includes a rectangular shape.

19. A method of containing and collecting particles produced during work, the method comprising the steps of:
providing a negative pressure enclosure, the enclosure including:
an enclosure having an outer shell defining an interior containment cavity and including a first opening at a rear side of the enclosure and at least one arm opening disposed within a front side of the enclosure providing access to the interior containment cavity;
a rear panel attached to the rear side and including a second opening aligned with the first opening;
a sealing portion including a first seal bordering the second opening and a second seal bordering the rear panel;
a vacuum pump port disposed through at least the rear panel; and
a vacuum hose coupler disposed through a side of the enclosure and into the interior containment cavity;
connecting a connector end of an air-line to the vacuum pump port, an opposite end of the air-line being connected to a vacuum pump;
turning the vacuum pump on;
placing the enclosure against a surface where said work is to be performed;
pushing the enclosure against the surface until the enclosure is held against the surface via a negative pressure created in the interior containment cavity by the vacuum pump;
inserting an end of a HEPA vacuum hose into the vacuum hose coupler, an opposite end of the HEPA vacuum hose being connected to a HEPA vacuum;
turning the HEPA vacuum on;
inserting at least one arm through the at least one arm opening; and
performing said work within the interior containment cavity, particles produced during said work being suctioned from the interior containment cavity via the HEPA vacuum hose, thereby maintaining an uncontaminated environment external to the enclosure.

20. The method of claim 19, wherein once said work is completed, the method further comprises the steps of:
turning the HEPA vacuum off;
cleaning out remaining particles left in the enclosure and on the surface; and
turning the vacuum pump off whilst simultaneously holding the enclosure until it comes loose from the surface.
